(19) United States
(12) Patent Application Publication
(10) Pub. No.: US 2008/0241828 A1
(43) Pub. Date: Oct. 2, 2008

(54) DETECTION OF DNA METHYLATION USING RAMAN SPECTROSCOPY

(76) Inventors: Kai Wu, Mountain View, CA (US); Xing Su, Cupertino, CA (US); Mineo Yamakawa, Campbell, CA (US)

Correspondence Address: INTEL/BSTZ;BLAKELY SOKOLOFF TAYLOR & ZAFMAN LLP, 1279 OAKMEAD PARKWAY, SUNNYVALE, CA, 94085-4040, US

(21) Appl. No.: 11/694,427

(22) Filed: Mar. 30, 2007

(51) Int. Cl. C12Q 1/68

(52) U.S. Cl. 435/6

(57) ABSTRACT

Epigenetic events such as DNA methylation play important roles in the regulation of gene expression. DNA methylation patterns have been found to differ between healthy and diseased tissue, such as healthy and cancerous tissue, thereby allowing DNA methylation to serve as a biomarker for disease states. Embodiments of the invention provide methods for detecting methylation patterns in DNA polymers. Methylation patterns are detected, in part, through the use of surface enhanced Raman spectroscopy (SERS). SERS provides a sensitive structure-based technique for chemical analysis.

### BACKGROUND OF THE INVENTION

1. Field of the Invention

Embodiments of the present invention relate generally to the detection of methylated nucleic acids, and to Raman spectroscopy.

2. Background Information

Although regulation of gene expression is a complex process, epigenetic events, such as the methylation of DNA, play important roles in the regulation of gene expression. DNA methylation in vivo is a post-synthetic modification associated with the silencing (or turning off) of genes. Mechanistically, DNA methylation has been associated with closed chromatin state (the three dimensional structure adopted by DNA in a cell nucleus) and therefore repressed or inactive genes. DNA methylation typically occurs at CpG sites or clusters of CpG called CpG islands. Epigenetic alterations, such as the methylation of DNA, modify genetic expression without changes to the DNA sequence.

Epigenetic traits can be inherited, yet are not based on a change in the DNA sequence of a gene. It has been found that DNA methylation patterns differ between healthy and diseased tissue thereby allowing DNA methylation to serve as a biomarker for disease states. Sensitive detection of DNA methylation or lack thereof in samples of DNA from patients may allow the early detection of disease. For example, in breast cancers DNA methylation is the most common cause of inactivation of the p16 gene, an important regulator of cell division. In addition, cells with a methylated p16 gene in substantial number of healthy, cancer-free women have been found suggesting that p16 methylation may be an important early event in the transition of normal breast cells into a pre-cancerous state. This data provides just one example of the possibility for the early detection of a disease based on the methylation of a portion of the genome. In many diseases, like cancer, early detection leads to improved outcomes for patients.

### BRIEF DESCRIPTION OF THE FIGURES

FIG. 1 provides a flow diagram generally illustrating a method according to an embodiment of the invention in which the methylation state (i.e., whether or not nucleotides comprising the DNA polymer have been functionalized with a methyl group) of a sample of DNA is determined.

FIG. 2 shows an exemplary methylated cytosine and an un-methylated cytosine base provided for comparison.

FIG. 3 diagrams the determination of a methylation state of a sample of DNA using SERS detection.

FIG. 4 diagrams a chemical reaction in which a cytosine nucleotide is converted to a uracil nucleotide.

FIG. 5 schematically describes a Raman spectrometer that can be used for SERS measurements.

FIGS. 6A, 6B, 6C, 6D, and 6E provide results from SERS experiments with representative DNA oligonucleotides.

### DETAILED DESCRIPTION OF THE INVENTION

Among the many analytical techniques that can be used for chemical analyses, surface-enhanced Raman spectroscopy (SERS) has proven to be a sensitive method. A Raman spectrum, similar to an infrared spectrum, consists of a wavelength distribution of bands corresponding to molecular vibrations specific to the sample being analyzed (the analyte). Raman spectroscopy probes vibrational modes of a molecule and the resulting spectrum, similar to an infrared spectrum, is fingerprint-like in nature. As compared to the fluorescent spectrum of a molecule which normally has a single peak exhibiting a half peak width of tens of nanometers to hundreds of nanometers, a Raman spectrum has multiple structure-related peaks with half peak widths as small as a few nanometers.

To obtain a Raman spectrum, typically a beam from a light source, such as a laser, is focused on the sample generating inelastically scattered radiation which is optically collected and directed into a wavelength-dispersive spectrometer. Although Raman scattering is a relatively low probability event, SERS can be used to enhance signal intensity in the resulting vibrational spectrum. Enhancement techniques make it possible to obtain a 10⁶ to 10¹⁴ fold increase in Raman signal intensity.

A polynucleotide can be RNA or DNA, and can be a gene or a portion thereof, a cDNA, a synthetic polydeoxyribonucleic acid sequence, or the like, and can be single stranded or double stranded, as well as a DNA/RNA hybrid. In various embodiments, a polynucleotide, including an oligonucleotide (for example, a probe or a primer) can contain nucleoside or nucleotide analogs, or a backbone bond other than a phosphodiester bond. In general, the nucleotides comprising a polynucleotide are naturally occurring deoxyribonucleotides, such as adenine, cytosine, guanine or thymine linked to 2′-deoxyribose, or ribonucleotides such as adenine, cytosine, guanine or uracil linked to ribose. However, a polynucleotide or oligonucleotide also can contain nucleotide analogs, including non-naturally occurring synthetic nucleotides or modified naturally occurring nucleotides. One example of an oligomeric compound or an oligonucleotide mimetic that has been shown to have good hybridization properties is referred to as a peptide nucleic acid (PNA). In PNA compounds, the sugar-backbone of an oligonucleotide is replaced with an amide containing backbone, for example an aminoethylglycine backbone. In this example, the nucleobases are retained and bound directly or indirectly to an aza nitrogen atom of the amide portion of the backbone. PNA compounds are disclosed in Nielsen et al., Science, 254:1497-15 (1991), for example.

The covalent bond linking the nucleotides of a polynucleotide generally is a phosphodiester bond. However, the covalent bond also can be any of a number of other types of bonds, including a thiodiester bond, a phosphorothioate bond, a peptide-like amide bond or any other bond known to those in the art as useful for linking nucleotides to produce synthetic polynucleotides. The incorporation of non-naturally occurring nucleotide analogs or bonds linking the nucleotides or analogs can be particularly useful where the polynucleotide is to be exposed to an environment that can contain nucleolytic activity, including, for example, a tissue culture medium or upon administration to a living subject, since the modified polynucleotides can be less susceptible to degradation.

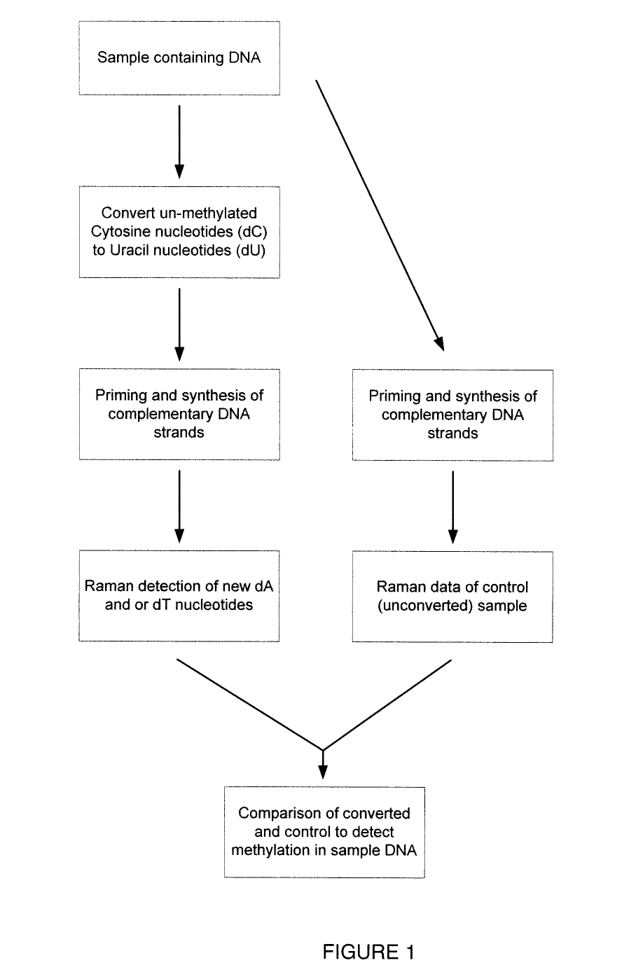

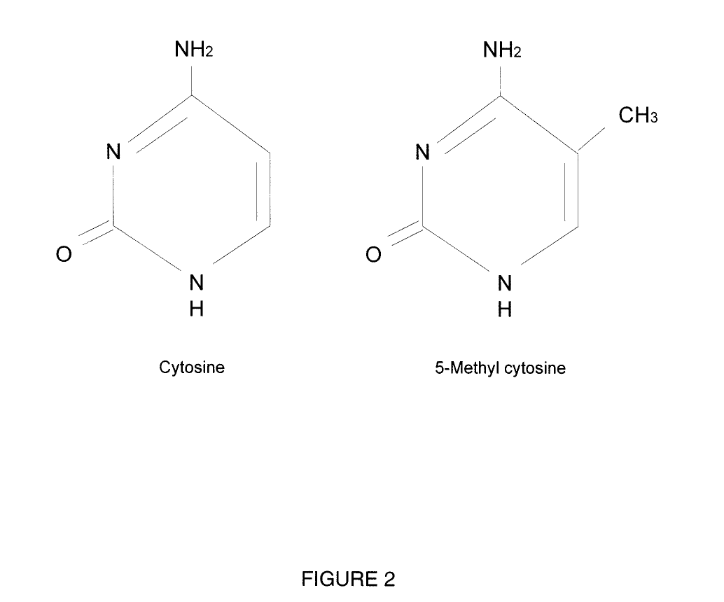

FIG. 1 describes a method for detecting the presence of methylated residues in a DNA polymer. FIG. 2 shows a cytosine base from a nucleotide and a 5-methyl cytosine base from a methylated nucleotide to demonstrate the site of methylation on cytosine residues. In the method of FIG. 1 a sample containing DNA to be analyzed, is divided into two portions. One portion of the DNA sample is treated to convert un-methylated cytosine residues (dC) to uracil residues (dU). The second portion of the sample containing DNA polymer to be analyzed remains unreacted. The DNA in both portions is then separately replicated thereby increasing the amount of DNA in each portion of the sample. Typically, DNA replication occurs through priming the DNA polymerase-mediated synthesis with the addition of short strands of DNA that are complementary to regions of the DNA in the sample. Polymerases are enzymes (nucleotidyltransferases) that catalyze the template-dependent synthesis of polynucleotide chains (DNA polymer) from deoxyribonucleoside triphosphates. The DNA polymer acts as a template for the synthesis of copies of itself. Primers can be random sequences of DNA or they can be specific for regions of interest on the DNA polymer, such as, for example, a region upstream of a known methylation site to be investigated. SERS spectra of the two sample portions are then obtained and compared. The presence of new residues (such as adenine (dA) or thymine (dT)) is indicative of cytosines that remained un-methylated in the DNA polymer sample. For genome-wide analysis procedures, complexity may optionally be reduced through the use of DNA methylation site-related restriction enzymes.

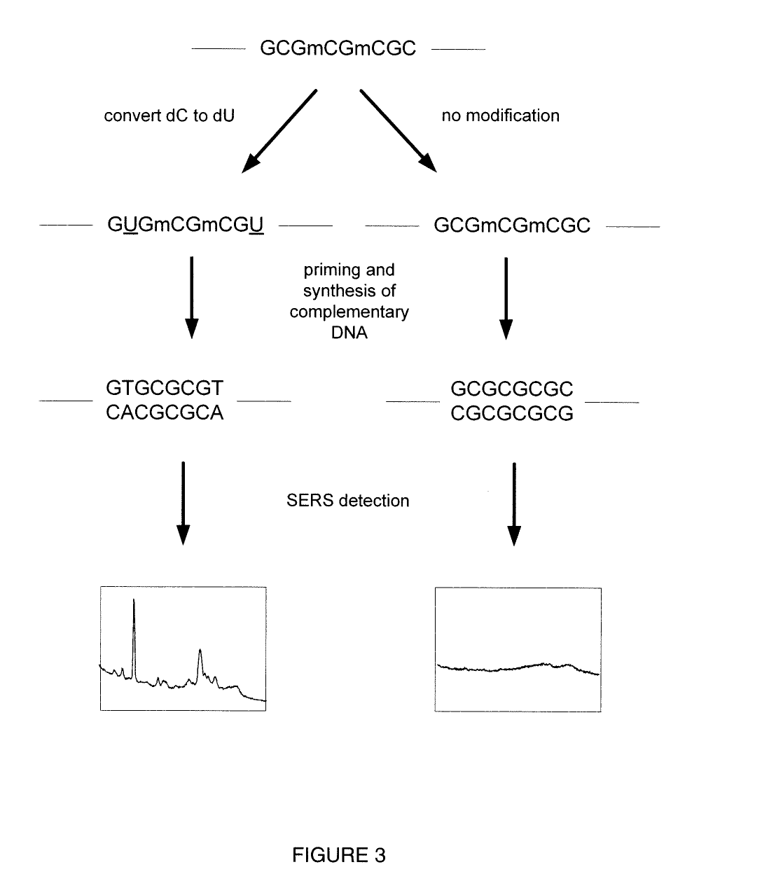

Referring now to FIG. 3, a method for determining the methylation state of an exemplary nucleic acid polymer is diagrammed. In the method of FIG. 3, a single-stranded DNA polymer having two methylated cytosines (indicated as “mC” in FIG. 3), undergoes chemical reaction to convert un-methylated cytosine nucleotides (dC) to uracil nucleotides (dU). A second sample of the single-stranded DNA polymer remains unreacted. Primers are added to both samples separately and complementary DNA is synthesized. In the complementary DNA that is synthesized the newly created uracil nucleotides are replaced by thymine nucleotides (dT) and on the complementary DNA strand an adenine nucleotide (dA) replaces the guanine (dG). The DNA samples are then separately associated with a SERS active surface and Raman spectroscopy is performed. In this relatively simple example, distinct differences are observed in the Raman spectra for the sample in which un-methylated cytosines have been converted to uracils because, in part, adenine nucleotides provide strong distinctive Raman signals. By comparing the converted sample with the unconverted sample of DNA polymer, a methylation state or pattern can be determined. Depending on the sample analyzed, the SERS spectra obtained from the samples may be more complicated and the detection of methylation pattern or state may be performed by pattern analysis. Since DNA methylation is often focused on CpG islands in the genomic sequence, the GC rich and often repeated CpG motif provide distinctive spectra and relatively stable background signatures. Optionally, DNA samples may also be fractionated by standard methods, for example, gel electrophoresis and HPLC (high performance liquid chromatography), and the fractions analyzed separately to obtain methylation information. In further embodiments, SERS spectra of the converted DNA polymer from the sample for which a methylation state is to be determined are compared with previously-obtained spectra of the methylated and or un-methylated DNA polymer having the same sequence or a sequence very similar to that of the sample of unknown-methylation-state DNA polymer, to determine the methylation state of the DNA in the sample.

In further embodiments of the present invention, priming and complementary DNA synthesis occurs in the presence of labeled nucleotides, such as, for example, labeled adenine or labeled thymine nucleotides, or labeled guanine or labeled cytosine nucleotides. In this case the labeled nucleotides are incorporated into the growing complementary DNA polymers. The labels are capable of providing distinctive Raman spectra. Thus, when the converted and the unconverted samples of DNA are associated with a Raman-active surface and Raman spectra are obtained, the distinctive spectra from the label molecules are visible. A comparison between the converted and the unconverted DNA polymer samples provides the ability to determine the methylation state of the DNA polymer. Some exemplary labeled nucleotides include, trinitrophenyl (TNP) labeled nucleotides, TMR labeled nucleotides, Texas Red labeled nucleotides, ROX labeled nucleotides, Rhodamine labeled nucleotides, Rhodamine Green labeled nucleotides, R6G labeled nucleotides, R110 labeled nucleotides, Fluorescein labeled nucleotides, Digoxigenin labeled nucleotides, Cy3 labeled nucleotides, Cy5 labeled nucleotides, Alexa Fluor labeled nucleotides, Aminonaphthalenesulfonate (AmNS) labeled nucleotides, BODIPY labeled nucleotides, caged nucleotides, Coumarin labeled nucleotides, fluorescein-12-dUTP, tetramethylrhodamine-6-dUTP, texas red-5-dUTP, lissamine-5-dUTP, diethylaminocoumarin-5-dUTP, cyanine-3-dUTP, cyanine-5-dUTP, fluorescein-12-dATP, and texas red-5-dATP. In general, any dye labeled dU (deoxy Uridine triphosphate), dT (deoxy Thymidine triphosphate), and dA (deoxy Adenine triphosphate) analogs are useful for labeling methylation site nucleotides. Suitable labeled nucleotides are, for example, commercially available from PerkinElmer Life and Analytical Science (Waltham, Mass., USA), Jena Bioscience (Jena, Germany), and Sierra Bioresearch (Phoenix, Ariz., USA).

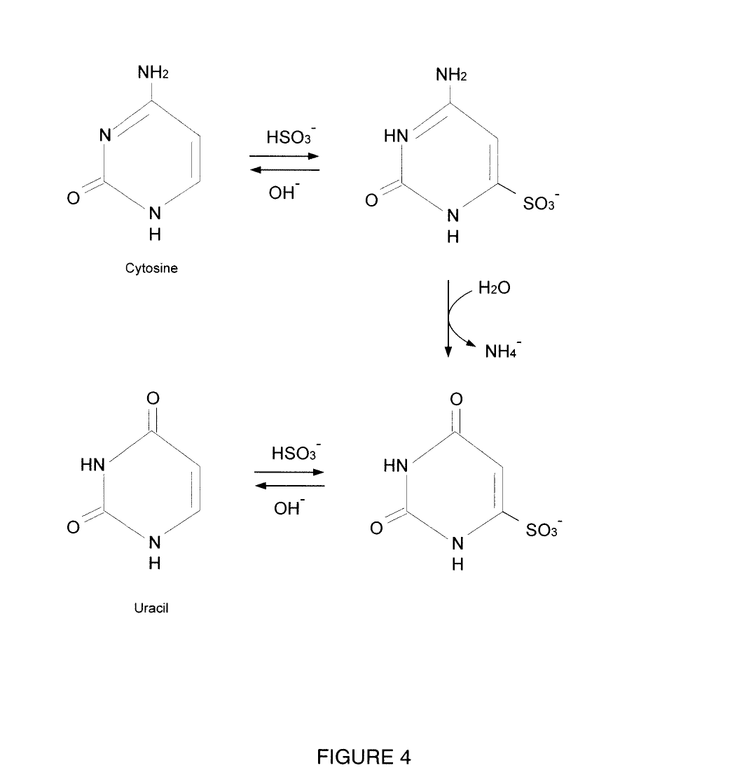

FIG. 4 diagrams a chemical reaction through which an un-methylated cytosine can be converted into a uracil. In this reaction, sodium bisulfite selectively modifies un-methylated cytosine and converts it to uracil. Methylated cytosines present in the reaction remain unchanged. In the reaction shown in FIG. 4, bisulfite is added to the 5-6 double bond of the cytosine base, the resulting cytosine bisulfite derivative is then hydrolytically deaminated yielding a uracil-bisulfite derivative, the uracil-bisulfate derivative then yields a uracil upon subsequent alkali treatment. Since the sulfonation reaction is favored in acidic solution, the reversible sulfonation reaction and the irreversible deamination reaction are carried out at a pH below 7. In the final step, the bisulfite adduct is removed from the uracil ring by alkali treatment (such as with NaOH, for example). See for example, Clark, S. J., Harrison, J., Paul, C. L., and Frommer, M., “High Sensitivity Mapping of Methylated Cytosines”, Nucleic Acids Research, 22, 2990-2997 (1994).

Optionally other nucleotide base modification procedures may be employed and a resulting change in DNA sequence detected with surface-enhanced Raman spectroscopy. For example, at neutral or alkaline pH, 5-methyl cytosine in DNA can be deaminated using high temperature, with grater rate than that of cytosine, resulting in the replacement of the 5-methyl cytosine with a thymine base. See for example, Wang, R. Y. et al., “Heat- and alkali-induced deamination of 5-methylcytosine residue in DNA,” Biochim. Biophys ACTA, 697:3, 371-7 (1982).

Further optionally, DNA methylation sites may be detected by using an enzymatic process other than the primer-extension process. For example, Uracil DNA Glycosylases (UDG) (an enzyme) removes dU from the DNA to form an apurinic/apyrimidinic site, which in turn can be modified by other DNA repair enzymes such as APE 1 and Enconuclease IV to form a free 3′-OH group. This free 3′-OH group can be further modified through terminal addition by terminal transferase (TdT) (an enzyme) incorporation of labeled nucleotide(s) or analogs. (UDG, TdT, APE1, and Endonuclease IV are commercially available, for example, from New England Biolabs-NEB, Ipswich, Mass., USA). The labeled nucleotide(s) or analogue(s) can then serve as detection target(s) for SERS analysis.

The nucleic acid analyte may be found directly in a sample such as a body fluid from a host. The sample can be examined directly or may be pretreated to render the analyte more readily detectible. The body fluid can be, for example, urine, blood, plasma, serum, saliva, semen, stool, sputum, cerebral spinal fluid, tears, mucus, and the like. In addition, the detection target can be any type of animal or plant cell, or unicellular organism. For example, an animal cell could be a mammalian cell such as an immune cell, a cancer cell, a cell bearing a blood group antigen such as A, B, D, etc., or an HLA antigen, or virus-infected cell. Further, the target cell could be a microorganism, for example, bacterium, algae, or protozoan.

Typically, a sample obtained from a biologic source, such as for example, a bodily fluid or cell lysate solution, is a complex mixture of proteins and other molecules. The components of the mixture can be separated using known techniques for isolating nucleic acid containing fractions, from biologic samples, such as for example, physical or affinity based separation techniques. The isolated nucleic acid fraction can then optionally be digested into smaller nucleic acids and or oligonucleotides. Typical methods include enzymatic digestions using, for example, type II restriction enzymes such as, AluI, BamHI, EcoRI, EcoRII, EcoRV, KpnI, NotI, HaeII, HindIII, BglI, and MboII. Restriction enzymes are endonucleases that cleave DNA polymers in response to a specific sequence of nucleotides, called restriction or recognition sites. DNA methylation site-related enzymes include, for example, McrBC endonuclease (commercially available from New England Biolabs-NEB, Ipswich, Mass., USA), which specifically recognizes methylation sites, and restriction endonucleases that are sensitive to methylation sites (and do not cut the DNA at the site), which include, AatII, AciI, AclI, AfeI, AscI, AsiSI, AvaI, BceAI, BmgBI, BasAI, BasHI, BsiEI, BsiWI, BsmBI, BspDI, BspEI, BsrBI, BsrFI, BssHII, BstBI, BstUI, ClaI, EagI, FauI, FseI, FspI, NotI, NGoMIV, NarI, NaeI, KasI, HpaII, HinPlI, SacII, SalI, SfoI, SmaI, SnaBI, and Xhol (among others) (commercially available from New England Biolabs-NEB, Ipswich, Mass., USA).

SERS Raman signal enhancement can occur through the association of the sample with a Raman active metal surface. Raman active surfaces of various forms can be used in embodiments of the present invention. For example, Raman active surfaces include, but are not limited to: a metallic surface, such as one or more layers of nanocrystalline and/or porous silicon coated with a metal or other conductive material; a particle, such as a metallic nanoparticle; an aggregate of particles, such as a metallic nanoparticle aggregate; a colloid of particles (with ionic compounds), such as a metallic nanoparticle colloid; or combinations thereof. Typical metals used for Raman enhancement include, silver, gold, platinum, palladium, copper, aluminum, zinc, iron or other conductive materials, although any metals capable of providing a SERS signal may be used. Especially large SERS enhancements have been observed with gold and silver surfaces. Additionally, the particles used to give SERS enhancements may be comprised of more than metal. For example, the particle could be silver coated gold or vice versa. The particles or colloid surfaces can be of various shapes and sizes. In various embodiments of the invention, nanoparticles of between 1 nanometer (nm) and 2 micrometers (μm) in diameter may be used. In alternative embodiments of the invention, nanoparticles of 2 nm to 1 μm, 5 nm to 500 nm, 10 nm to 200 nm, 20 nm to 100 nm, 30 nm to 80 nm, 40 nm to 70 nm or 50 nm to 60 nm diameter may be used. In certain embodiments of the invention, nanoparticles with an average diameter of 10 to 50 nm, 50 to 100 nm or about 100 nm may be used.

Substrate materials and or layer(s) used in embodiments of the invention may be porous or non-porous. For example, a substrate may be comprised of porous silicon. Further, substrates, including porous substrates, may be coated with a SERS-active metal layer in order to, for example, enhance SERS detection. Suitable porous materials include porous silicon (e.g., single crystal porous silicon), porous polysilicon, porous ceramics (e.g., those made from fibrous porous silicon nitride), porous silica, porous alumina, porous silicon-germanium, porous germanium, porous gallium arsenide, porous gallium phosphide, porous zinc oxide, and porous silicon carbide. Methods of making such porous materials are generally known. See, for example, Dougherty et al. (2002) Mat. Res. Soc. Symp. Proc. 687:B.7.3.1-B.7.3.6 (porous polysilicon), Ohji (2001) AIST Today 1:28-31 (porous ceramics), Trau et al. (1997) Nature 390:674-676 (porous silica), Masuda et al. (1995) Science 268:1466-1468 (porous alumina), Li et al. (1999) Adv. Mater. 11:483-487 (porous alumina), Nielsch et al. (2000) Adv. Mater. 12:582-586 (porous alumina), Buttard et al. (1997) Thin Solid Films 297:233-236 (porous silicon-germanium), van Vugt et al. (2002) Chem Commun. 2002:2054-2055 (porous germanium), Kamenev et al. (2000) Semiconductors 34:728-731 (porous gallium arsenide), Buzynin et al. (2000) Tech. Physics 45:650-652 (porous gallium arsenide), Shuurmans et al. (1999) Science 284:141-143 (porous gallium phosphide), Lubberhuizen et al. (2000) J. Porous Mat. 7:147-152 (porous gallium phosphide), Terada et al. (1999) 4th Int'l. Conf. on Ecomaterials P-30:559-562 (porous zinc oxide), Jessensky et al. (1997) Thin Solid Films 297:224-228 (porous silicon carbide), Spanier et al. (2000) Appl. Phys. Lett. 76:3879-3881 (porous silicon carbide), Spanier et al. (2000) Physical Review B 61:10437-10450 (porous silicon carbide), and Sangsig et al. (2000) Jpn. J. Appl. Phys. 39:5875-5878 (porous silicon carbide). The substrate can include a plurality of layers of the porous material.

Porous silicon is a material that can be made simply and inexpensively. As observed by high resolution scanning and transmission electron microscope, porous silicon typically has pore diameters varying from a few nanometers to several micrometers, depending upon the conditions under which the porous silicon was formed. The term “porous” as used herein may be defined consistent with the IUPAC guidelines, wherein “microporous” refers to pores having a size regime that is less than or equal to two nanometers (nm), “mesoporous” refers to pores having a size regime that is between about 2 and 50 nm, and “macroporous” refers to pores having a size regime that is greater than about 50 nm. See e.g., Cullis et al. (1997) J. Appl. Phys. Rev. 82:909-965. Porous materials, such as porous silicon, may be made by many different techniques, the most common of which is one using electrochemistry because a relatively large and relatively homogeneous substrate can be readily formed by such technique. While porous silicon substrates can be prepared by a variety of techniques, such as, for example, stain etching and anodic etching, preferably, porous silicon substrates are prepared by anodic electrochemical etching. Anodic electrochemical etching permits control of properties of the formed substrate such as, for example, microstructure, pore diameter, porosity, refractive index, and thickness. Anodic electrochemical etching includes immersing an electrode (e.g., a platinum electrode) and a silicon wafer in an electrolytic bath containing, for example, water, ethanol, and hydrofluoric acid (HF), or solutions of hydrogen nitrate (HNO₃) in HF. While in solution, the wafer is subjected to a constant current in a range of about 1 mA/cm² to about 1000 mA/cm². The current is applied to the wafer for a time period ranging from several seconds to several hours, preferably for up to about one hour, to form a layer of porous silicon at or on the surface of the wafer. Etching and anodization can occur with or without illumination depending upon the type of substrate dopant.

Surface enhanced Raman spectroscopy is performed on a sample by, for example, mixing the sample with a SERS solution comprising a Raman active surface, such as for example, colloidal silver metal particles; depositing and drying the digested sample onto a substrate and subsequently adding a SERS solution, such as a colloidal silver solution; depositing the sample onto a SERS-active substrate; or it can be performed in-line in a component of a microfluidic or nanofluidic system, such as by using a micro or nanomixer to mix the SERS solution with a the sample and subsequently performing Raman analysis on the sample. A silver colloidal solution can be mixed with sample eluants in a fluidic format, optionally, on a chip using microfluidics, and the detection can be performed inline as the eluants are flowing through the laser detection volume. In additional embodiments, some or all of these steps are performed using microfluidic or nanofluidic systems. Optionally, Raman enhancements may be achieved through the use of lithium chloride (LiCl) in conjunction with the Raman active metal surface. For example, lithium chloride may be added to a silver nanoparticle solution at a final concentration of 0.18 M and the silver nanoparticle solution placed in contact with the DNA solution in order to enhance the Raman signal from the nucleic acids. See for example, U.S. Pat. No. 7,019,828, entitled “Chemical Enhancement in Surface Enhanced Raman Scattering Using Lithium Salts.”

Micro or nanofluidic analytical systems (lab-on-a-chip type devices) typically are created from a network of channels and reservoirs (or wells) formed in a substrate. Nanofluidics refers to devices having channels that are about 100 to 1000 times smaller than microfluidic channels. Typical substrates include, for example, glass, quartz, polymers, especially biocompatible polymers, such as for example polydimethylsiloxane (PDMS), polystyrene, polyethylene, metals, silicon, silicon nitride, and silicon oxide, although any machinable, etchable, reformable, moldable, stampable, embossable, or castable elastomeric material (a material that is capable of deforming when pressure is applied and returning to its original shape when pressure is removed) may potentially be used for all or some part(s) of the device. Fluid may be moved through the chip by a variety of mechanisms, including electrokinetic or electroosmotic forces, pumps, peristaltic pumps, gravity, injectors, syringes, and membrane-actuated pumps. Lab-on-a-chip devices may perform operations such as, for example, sample handling, mixing, dilution, eletrophoretic, chromatographic, and size-based separations, molecular labeling and detection. Because the volume of fluids within microchannels is very small, usually several nanoliters or less, the amount of reagents and analytes used is small.

In the practice of embodiments of the present invention, a Raman spectrometer can be part of a detection unit designed to detect and quantify phosphopeptides labeled with Raman tags by Raman spectroscopy. Methods for detection of Raman labeled analytes, for example nucleotides, using Raman spectroscopy are known in the art. See, for example, U.S. Pat. Nos. 5,306,403; 6,002,471; and 6,174,677. A non-limiting example of a Raman detection unit is disclosed in U.S. Pat. No. 6,002,471. An excitation beam is generated by either a frequency doubled Nd:YAG laser at 532 nm wavelength or a frequency doubled Ti:sapphire laser at 365 nm wavelength. Pulsed laser beams or continuous laser beams may be used. The excitation beam passes through confocal optics and a microscope objective, and is focused onto the flow path and/or the flow-through cell. The Raman emission light from the labeled nanoparticles is collected by the microscope objective and the confocal optics and is coupled to a monochromator for spectral dissociation. The confocal optics includes a combination of dichroic filters, barrier filters, confocal pinholes, lenses, and mirrors for reducing the background signal. Standard full field optics can be used as well as confocal optics. The Raman emission signal is detected by a Raman detector, which includes an avalanche photodiode interfaced with a computer for counting and digitization of the signal.

Another example of a Raman detection unit is disclosed in U.S. Pat. No. 5,306,403, including a Spex Model 1403 double-grating spectrophotometer with a gallium-arsenide photomultiplier tube (RCA Model C31034 or Burle Industries Model C3103402) operated in the single-photon counting mode. The excitation source includes a 514.5 nm line argon-ion laser from SpectraPhysics, Model 166, and a 647.1 nm line of a krypton-ion laser (Innova 70, Coherent).

Alternate excitation sources include a nitrogen laser (Laser Science, Inc.) at 337 nm and a helium-cadmium laser (Liconox) at 325 nm (U.S. Pat. No. 6,174,677), a light emitting diode, an Nd:YLF laser, and/or various ions lasers and/or dye lasers. The excitation beam may be spectrally purified with a bandpass filter (Corion) and may be focused on the flow path and/or flow-through cell using a 6× objective lens (Newport, Model L6X). The objective lens may be used to both excite the Raman-active organic compounds and to collect the Raman signal, by using a holographic beam splitter (Kaiser Optical Systems, Inc., Model HB 647-26N18) to produce a right-angle geometry for the excitation beam and the emitted Raman signal. A holographic notch filter (Kaiser Optical Systems, Inc.) may be used to reduce Rayleigh scattered radiation. Alternative Raman detectors include an ISA HR-320 spectrograph equipped with a red-enhanced intensified charge-coupled device (RE-ICCD) detection system (Princeton Instruments). Other types of detectors may be used, such as Fourier-transform spectrographs (based on Michaelson interferometers), charged injection devices, photodiode arrays, InGaAs detectors, electron-multiplied CCD, intensified CCD and/or phototransistor arrays.

Any suitable form or configuration of Raman spectroscopy or related techniques known in the art may be used for detection of DNA, including but not limited to normal Raman scattering, resonance Raman scattering, surface enhanced Raman scattering, surface enhanced resonance Raman scattering, coherent anti-Stokes Raman spectroscopy (CARS), stimulated Raman scattering, inverse Raman spectroscopy, stimulated gain Raman spectroscopy, hyper-Raman scattering, molecular optical laser examiner (MOLE) or Raman microprobe or Raman microscopy or confocal Raman microspectrometry, three-dimensional or scanning Raman, Raman saturation spectroscopy, time resolved resonance Raman, Raman decoupling spectroscopy or UV-Raman microscopy.

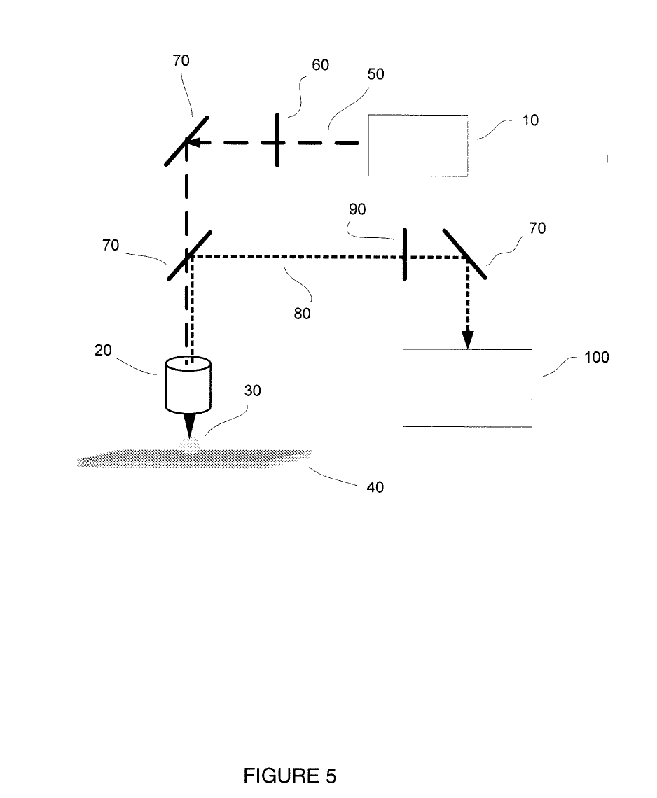

FIG. 5 shows a schematic of a Raman spectrometer setup that was used for the SERS measurements. The system consisted of a titanium:sapphire laser 10 (Mira by Coherent, Santa Clara, Calif.) operating at 785 nm with power levels of about 750 mW, and a 20× microscope objective 20 (Nikon LU series) to focus the laser spot onto the sample plane. The sample 30 was placed on a substrate 40. The excitation beam 50 was filtered by a dielectric filter 60 (Chroma Technology Corp., Brattleboro, Vt.), to suppress spontaneous emission from the laser and reflected from a dichroic mirror 70 (Chroma Technology Corp., Brattleboro, Vt.). The Raman scattered light 80 from the sample 30 was collected by the same microscope objective 20, and was reflected off the dichroic mirror 70 toward a notch filter or bandpass filter 90 (Kaiser Optical Systems, Ann Arbor, Mich.). The notch filter 90 blocked the laser beam and transmitted Raman scattered light 80. The Raman-scattered light was imaged onto the slit of a spectrophotometer 100 (Acton Research Corp., Acton, Mass.) (using dichroic mirror 70) that was connected to a thermo-electrically cooled charge-coupled device (CCD) detector (Princeton Instruments, Princeton, N.J.) (not shown). The CCD camera was connected to a PC (not shown), and the collected spectrum was transported to the PC for visual display and computational analysis.

### EXAMPLE

In the following example, model oligonucleotides that allowed the comparison of a methylated CpG sequence with a non-methylated CpG sequence were detected by SERS. The first sequence was a self-annealing control sequence: CGCGCGCGCGCGCGCGCGCG (GC_control). The second sequence was designed to mimic the single methylase product CGCG to UGUG and its complementary strand. The second sequence was CGCGCGCGUGUGCGCGCGCG (U1) and CGCGCGCGCACACGCGCGCG (A1) where U1 and A1 form a duplex after annealing. The third sequence was UGUGUGUGUGUGUGUGUGUG (U_all) and its complement CACACACACACACACACACA (A_all) (also forming a duplex after annealing).

The experimental procedures were as follows:

Annealing reaction to form duplex: Concentrated oligos (50 or 100 μM) for annealing pair or self-annealing were prepared in Tris buffer containing 50 mM NaCl, boiled for 10 minutes, and cooled gradually to room temperature.

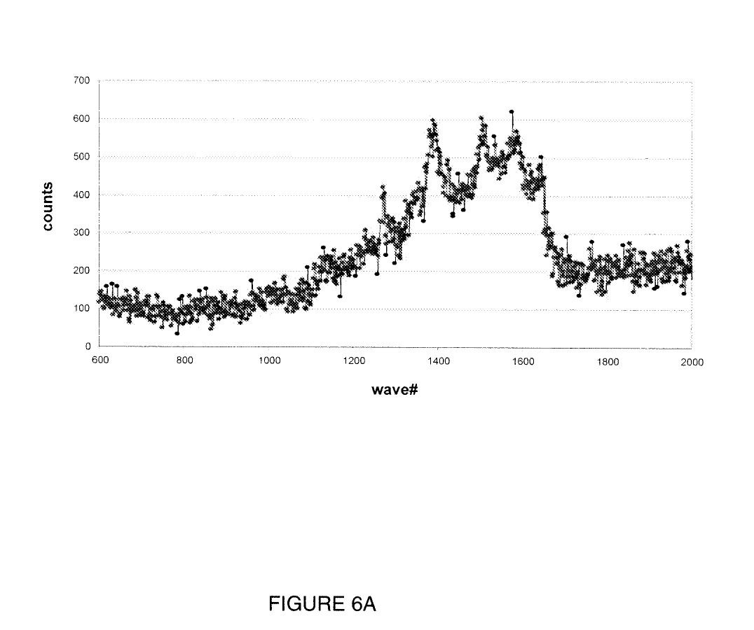

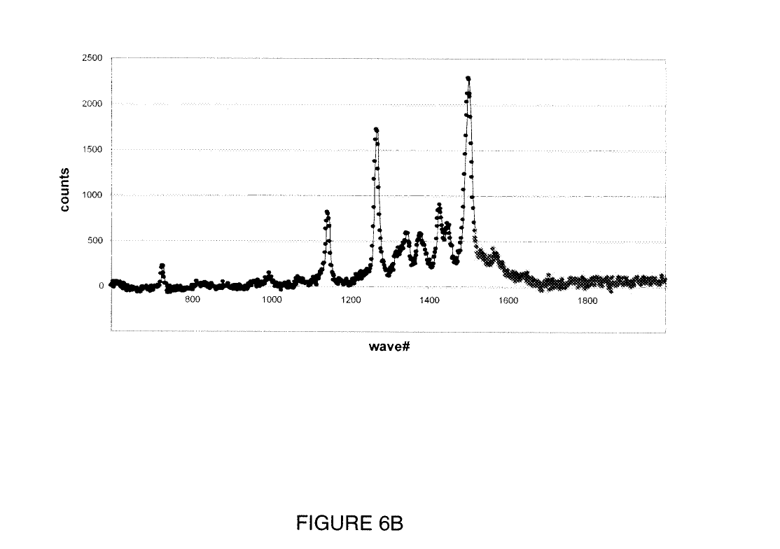

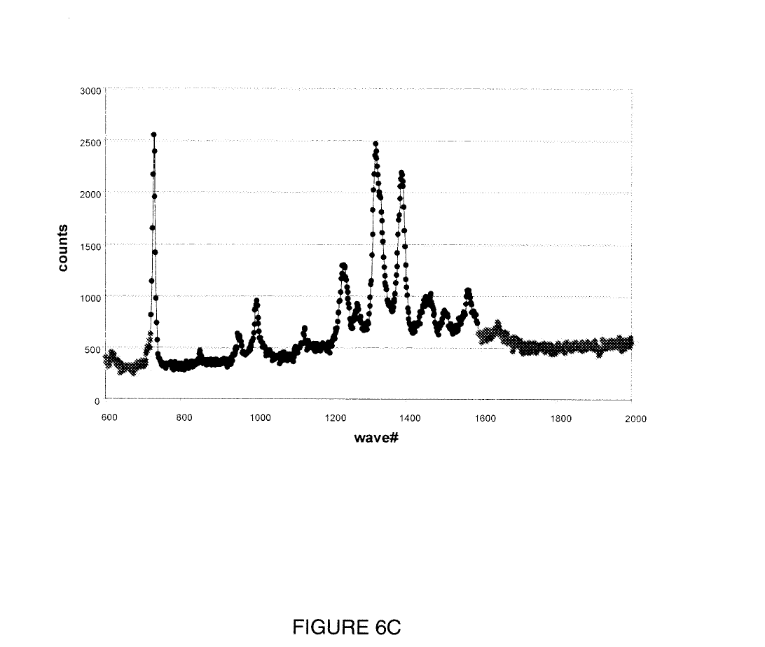

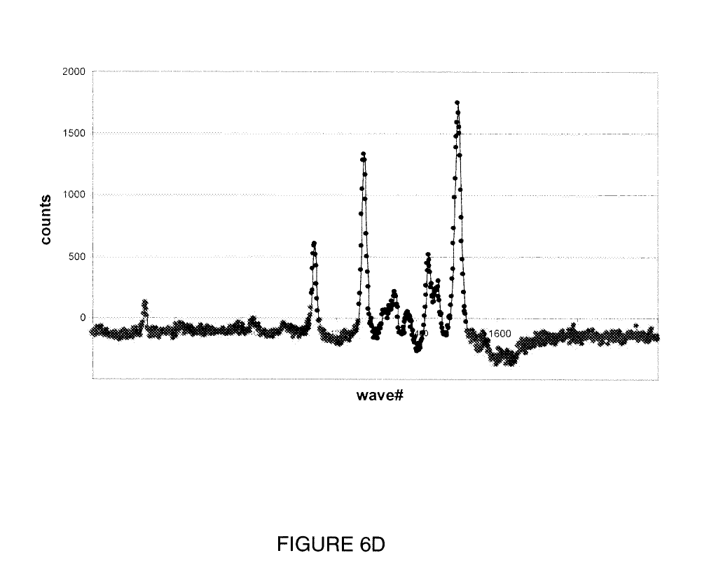

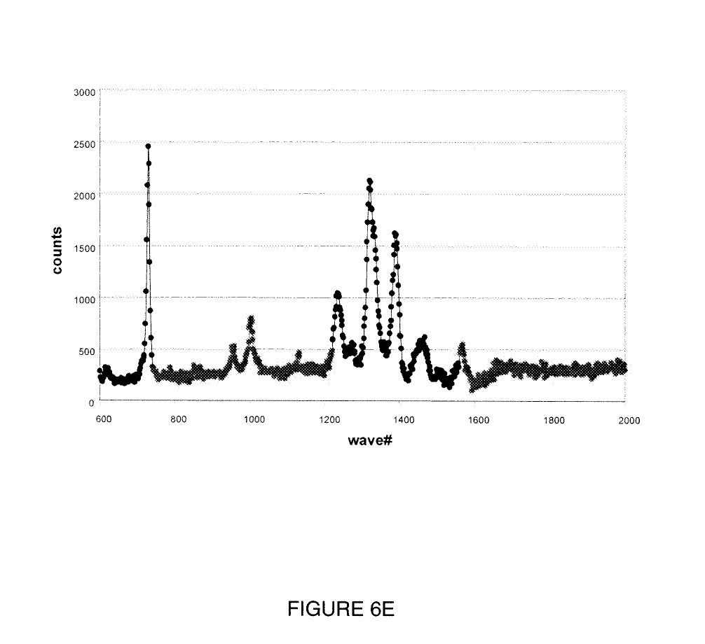

SERS experiment: 20 μM of annealed duplex oligonucleotides, in final concentration of 50 mM NaCl, was mixed with Ag colloid (0.15 M) for 1 minute, then acetylated-BSA (50 mg/ml) was added to stop the aggregation. After 1-3 minutes, SERS measurements with replicates were taken and analyzed. For each presented data, the background (solution with identical concentration and components minus oligonucleotide duplex) was subtracted from the measurement. In paired analysis, direct subtraction was made between the test pair duplex and GC_control duplex. FIG. 6A provides the SERS spectrum of the GC_control (20 μM) with the background subtracted. FIG. 6B provides the SERS spectrum of the U1:A1 duplex (20 μM) with the background subtracted. FIG. 6C provides the SERS spectrum of the U_all:A_all duplex with the background subtracted. FIG. 6D provides the SERS spectrum of the U1:A1 duplex (20 μM) with the GC_control spectrum subtracted. FIG. 6E provides the SERS spectrum of the U_all:A_all duplex with the with the GC_control spectrum subtracted. As can be seen from the figures, subtraction of the control sequence spectrum (FIGS. 6D and 6E) provides a distinctive spectral result indicating the presence of dU and dA nucleotides in the test samples.

What is claimed is:

1. A method for analyzing a sample containing DNA polymer comprising:
converting one or more cytosine nucleotides in the DNA polymer in the sample to uracil nucleotides;
increasing the amount of DNA polymer in the sample by synthesizing DNA polymer that is complementary to the DNA polymer in the sample;
obtaining a surface enhanced Raman spectrum of the DNA polymer in the sample by associating the DNA polymer in the sample with a surface capable of enhancing the Raman signal obtained from the DNA polymer; and
determining the presence or absence of methylated cytosine nucleotides within the DNA polymer in the sample.

2. The method of claim 1 wherein increasing the amount of DNA polymer in the sample by synthesizing DNA polymer that is complementary to the DNA polymer in the sample occurs after converting one or more cytosine nucleotides in the DNA polymer in the sample to uracil nucleotides.

3. The method of claim 1 wherein increasing the amount of DNA polymer in the sample by synthesizing DNA polymer that is complementary to the DNA polymer in the sample is accomplished by randomly priming the synthesis of DNA polymer.

4. The method of claim 1 wherein increasing the amount of DNA polymer in the sample by synthesizing DNA that is complementary to the DNA polymer in the sample is accomplished by selectively priming the synthesis of complementary DNA polymer using primers that are selective for some and for not all the sequence of the DNA polymer in the sample.

5. The method of claim 1 wherein the surface capable of enhancing the Raman signal from the DNA polymer is a metal surface.

6. The method of claim 1 wherein the surface capable of enhancing the Raman signal from the DNA polymer is a metal surface and the metal is selected from the group consisting of aluminum, copper, silver, gold, platinum, palladium, zinc, iron, and combinations thereof.

7. The method of claim 1 wherein the surface capable of enhancing the Raman signal from the DNA polymer is a porous silicon surface having a metal layer.

8. The method of claim 1 wherein determining the presence or absence of methylated nucleotides within the DNA polymer in the sample comprises comparing the surface enhanced Raman spectrum of the DNA polymer in the sample in which one or more cytosine nucleotides have been converted to the surface enhanced Raman spectrum of the DNA polymer in which no cytosine nucleotides have been converted.

9. The method of claim 1 wherein the sample is derived from cellular material from an organism.

10. A method for analyzing a sample containing DNA polymer comprising:
converting one or more cytosine nucleotides in the DNA polymer in the sample to uracil nucleotides;
increasing the amount of DNA polymer in the sample by synthesizing DNA polymer that is complementary to the DNA polymer in the sample wherein the synthesis occurs in the presence of one or more labeled nucleotides under conditions that allow the labeled nucleotides to become incorporated into the synthesized DNA polymer and wherein the labeled nucleotides are capable of providing distinctive surface enhanced Raman spectra;
obtaining a surface enhanced Raman spectrum of the DNA polymer in the sample by associating the DNA polymer in the sample with a surface capable of enhancing the Raman signal obtained from the DNA polymer molecule; and
determining the presence or absence of labeled adenine nucleotides within the DNA polymer in the sample by detecting the presence of a labeled nucleotide.

11. The method of claim 10 in which the labeled nucleotides are labeled adenine or labeled thymine nucleotides.

12. The method of claim 10 wherein increasing the amount of DNA polymer in the sample by synthesizing DNA polymer that is complementary to the DNA polymer in the sample occurs after converting one or more cytosine nucleotides in the DNA polymer in the sample to uracil nucleotides.

13. The method of claim 10 wherein increasing the amount of DNA polymer in the sample by synthesizing DNA polymer that is complementary to the DNA polymer in the sample is accomplished by randomly priming the synthesis of DNA polymer.

14. The method of claim 1 wherein increasing the amount of DNA polymer in the sample by synthesizing DNA that is complementary to the DNA polymer in the sample is accomplished by selectively priming the synthesis of complementary DNA polymer using primers that are selective for some and for not all the sequence of the DNA polymer in the sample.

15. The method of claim 10 wherein the surface capable of enhancing the Raman signal from the DNA polymer is a metal surface.

16. The method of claim 10 wherein the surface capable of enhancing the Raman signal from the DNA polymer is a metal surface and the metal is selected from the group consisting of aluminum, copper, silver, gold, platinum, palladium, zinc, iron, and combinations thereof.

17. The method of claim 10 wherein the surface capable of enhancing the Raman signal from the DNA polymer is a porous silicon surface having a metal layer.

18. The method of claim 10 wherein determining the presence or absence of methylated nucleotides within the DNA polymer in the sample comprises comparing the surface enhanced Raman spectrum of the DNA polymer in the sample in which one or more cytosine nucleotides have been converted to the surface enhanced Raman spectrum of the DNA polymer in which no cytosine nucleotides have been converted.

19. The method of claim 10 wherein the sample is derived from cellular material from an organism.


SEQUENCE LISTING (9 sequences)

SEQ ID NO: 1 - DNA, 20 bp, Artificial Sequence
  Synthetic sequence
cgcgcgcgcgcgcgcgcgcg

SEQ ID NO: 2 - DNA, 20 bp, Artificial Sequence
  Synthetic sequence
cgcgcgcgugugcgcgcgcg

SEQ ID NO: 3 - DNA, 20 bp, Artificial Sequence
  Synthetic sequence
cgcgcgcgcacacgcgcgcg

SEQ ID NO: 4 - DNA, 20 bp, Artificial Sequence
  Synthetic sequence
ugugugugugugugugugug

SEQ ID NO: 5 - DNA, 20 bp, Artificial Sequence
  Synthetic sequence
cacacacacacacacacaca

SEQ ID NO: 6 - DNA, 4 bp, Artificial Sequence
  Synthetic sequence
cgcg

SEQ ID NO: 7 - DNA, 4 bp, Artificial Sequence
  Synthetic sequence
ugug

SEQ ID NO: 8 - DNA, 8 bp, Artificial Sequence
  Synthetic sequence
gcgcgcgc

SEQ ID NO: 9 - DNA, 8 bp, Artificial Sequence
  Synthetic sequence
gtgcgcgt